(19) United States
(12) Patent Application Publication
(10) Pub. No.: US 2018/0116591 A1
(43) Pub. Date: May 3, 2018

(54) Medical Information Bracelet

(71) Applicant: Kimberly Hensley, Hohenwald, TN (US)

(72) Inventor: Kimberly Hensley, Hohenwald, TN (US)

(21) Appl. No.: 15/494,070

(22) Filed: Apr. 21, 2017

Related U.S. Application Data
(60) Provisional application No. 62/416,880, filed on Nov. 3, 2016.

(51) Int. Cl. A61B 5/00; A44C 5/00; A61B 5/0205

(52) U.S. Cl. CPC A61B 5/6802; A44C 5/0023; A44C 5/0015; A61B 5/021; A61B 5/742; A61B 5/0205; A44C 5/0069

(57) ABSTRACT

A medical information bracelet for storing and retrieving medical information from a user. The medical information bracelet includes a housing disposed on an elongated strap, wherein the housing comprises a visual display screen and a non-transitory computer readable medium. A sensor configured to detect physiological data, wherein the sensor is in electronic communication with the visual display screen. A port disposed on the strap and operably connected to the computer readable medium, wherein the port is configured to receive medical information. A logic that is at least partially stored in the non-transitory computer readable medium and that, when executed at least in part by a processor, causes the medical information bracelet to perform a method, the method comprising: transmitting the physiological data detected from the sensor to the screen, such that the screen displays blood pressure, heart rate, and other types of physiological data; receiving medical information received by the port.

### CROSS REFERENCE TO RELATED APPLICATIONS

This application claims the benefit of U.S. Provisional Application No. 62/416,880 filed on Nov. 3, 2016. The above identified patent application is herein incorporated by reference in its entirety to provide continuity of disclosure.

### BACKGROUND OF THE INVENTION

The present invention relates to medical information bracelets. More specifically, the present invention includes a medical information bracelet that stores and retrieves medical information from the user.

Electronic medical records are becoming more and more prominent in the medical industry. When a person has multiple doctors and specialists, it can be difficult for one doctor to view a complete medical record of the patient if they are unable to coordinate with the other doctors. Patients generally do not have simple and effective means to manage their own medical records while having it with them. A patient may have a personal medical record but does not wear it or bring it with them and therefore it is left behind when an emergency occurs. In addition, a medical professional or emergency responder is unable to locate it when the patient is unconscious or suffers from memory and/or cognitive problems. Medical providers are challenged by obtaining medical records often due to records being electronic or paper which creates difficulty in forwarding necessary documentation to the medical professional requesting such data. Therefore, there exists a need for a device that can quickly supply doctors with a patient's medical history and information.

### SUMMARY OF THE INVENTION

In view of the foregoing disadvantages inherent in the known types of medical information bracelets now present in the prior art, the present invention provides a new medical information bracelet wherein the same can be utilized for providing convenience for the user when storing and retrieving medical information from the user.

The present system comprises a housing disposed on an elongated strap, wherein the housing comprises a visual display screen and a non-transitory computer readable medium. A sensor configured to detect physiological data, wherein the sensor is in electronic communication with the visual display screen. A port disposed on the strap and operably connected to the computer readable medium, wherein the port is configured to receive medical information. A logic that is at least partially stored in the non-transitory computer readable medium and that, when executed at least in part by a processor, causes the medical information bracelet to perform a method, the method comprising: transmitting the physiological data detected from the sensor to the screen, such that the screen displays blood pressure, heart rate, and other types of physiological data; receiving medical information received by the port.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a front perspective view of the medical information bracelet.

FIG. 2 shows a cross-sectional view of the medical information bracelet.

FIG. 3 shows a top view of the medical information bracelet.

FIG. 4 shows a bottom view of the medical information bracelet.

FIG. 5 shows a perspective view of an alternate embodiment of the medical information bracelet.

FIG. 6 shows a block diagram of the electronic components of the medical information bracelet.

### DETAILED DESCRIPTION OF THE INVENTION

Reference is made herein to the attached drawings. Like reference numerals are used throughout the drawings to depict like or similar elements of the medical information bracelet. The figures are intended for representative purposes only and should not be considered to be limiting in any respect.

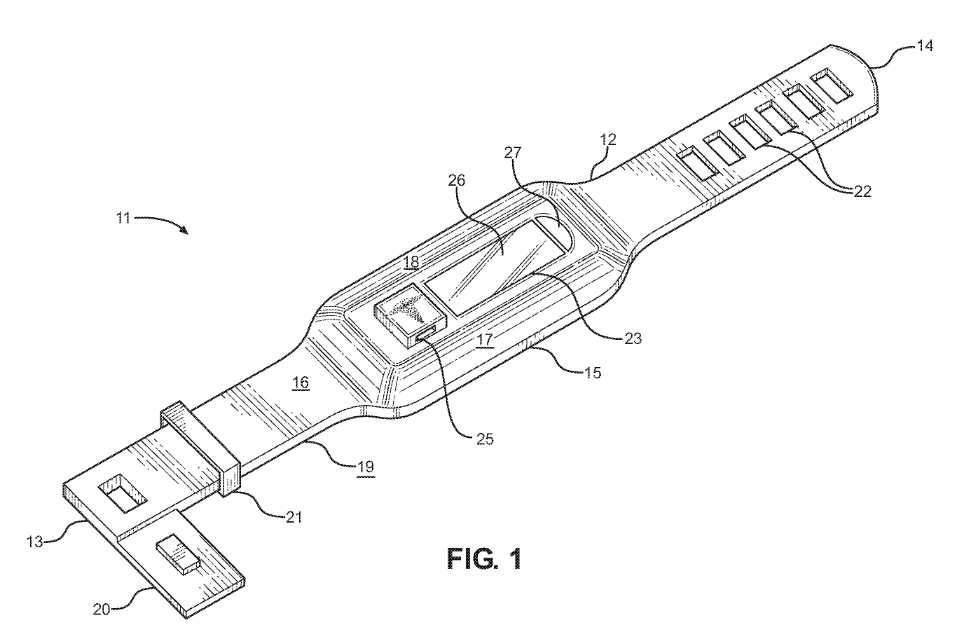

Referring now to FIG. 1, there is shown a front perspective view of the medical information bracelet. The medical information bracelet 11 comprises an elongated strap 12 including a first end 13, a second end 14, and a middle section 15. The elongated strap 12 is a uniform material throughout and is constructed from rubber, silicon, or a similar material. The medical information bracelet 11 is adapted to store medical information within the elongated strap 12. In an alternate embodiment, the medical information is stored and worn on the user's neck as a necklace.

The elongated strap 12 includes a first surface 16, a second surface 17, a third surface 18, and a fourth surface 19. In the illustrated embodiment, the first and fourth surfaces 16, 19 each extend the entire length of the elongated strap 12 from the distal end of the first end 13 to the distal end of the second end 14. The first surface 16 and the fourth surface 19 are parallel to each other. The second surface 17 creates an acute angle to the first surface 16 at the middle section 15. The second surface 17 extends the entire length of the middle section 15. The third surface 18 creates an acute angle to the first surface 16 at the middle section 15. The third surface 18 extends the entire length of the middle section 15 and is on an opposite side to the second surface 17.

The first end 13 comprises a fastener 20 and a strap keeper 21. In the illustrated embodiment, the fastener 20 is a locking mechanism located at the distal end of the first end 13 adapted to secure the medical information bracelet 11 in a loop formation. However, in alternate embodiments, the fastener 20 is a strap buckle clasp or a similar type of locking mechanism. The strap keeper 21 is located between the fastener 20 and the middle section 15. The strap keeper 21 retains the second end 14 in place once it has been fastened.

The second end 14 comprises a plurality of apertures 22. The plurality of apertures 22 are for receiving the fastener 20. In the illustrated embodiment, the plurality of apertures 22 are located at the distal end of the second end 14 for adjusting the size of the elongated strap 12 around a user's wrist. The apertures 22 are aligned side-by-side along the length of the second end 14 of the elongated strap 12. The opening on the first end 13 aligned with the fastener 20 is adapted to align with the apertures 22. Once aligned, the fastener 20 is positioned through the aperture 22 and the opening on the first end 13.

The middle section 15 comprises a housing 23, a first port 25, a visual display screen 26, and a function button 27. In the illustrated embodiment, the middle section 15 is wider than the first end 13 and the second end 14, wherein width is measured from the second surface 17 to the third surface 18. The middle section 15 is wider to allow for the visual display screen 26 to vary in size. However, in alternate embodiments, the middle section 15 comprises the same width as the first end 13 and the second end 14. In the illustrated embodiment, the middle section 15 is rectangular in shape. However, in alternate embodiments, the middle section 15 is any suitable shape, such as circular.

In the illustrated embodiment, the first port 25 is positioned on the first surface 16 of the elongated strap 12, adjacent to the visual display screen 26. The function button 27 is positioned on an opposing side of the visual display screen 26, such that the first port 25, the visual display screen 26, and the function button 27 are aligned side by side.

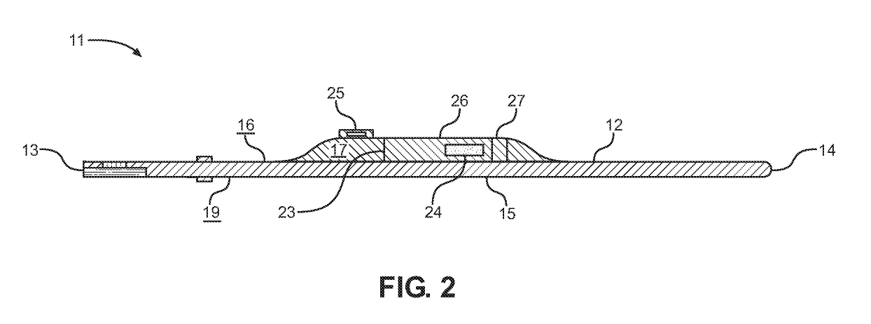

Referring now to FIG. 2, there is shown a cross-sectional view of the medical information bracelet. The second surface 17 extends the entire length of the middle section 15 and begins and ends where the elongated band 12 becomes thicker. The middle section 15 is thicker than the first end 13 and the second end 14, wherein thickness is measured from the first surface 16 to the fourth surface 19, to allow for the housing to hold all the necessary electrical components.

The housing 23 comprises a storage medium 24, the visual display screen 26, and the function button 27. In the illustrated embodiment, the housing 23 is slightly off centered in the middle section 15. However, in alternate embodiments, the housing 23 is disposed in the center. The storage medium 24 is embedded in the housing 23 adapted to be in electronic communication with the visual display screen 26 and function button 27. The visual display screen 26 is adapted to completely cover the upper surface of the housing 23. In the illustrated embodiment, the storage medium 24 is a microchip adapted to place, retrieve, and store data measured by a physiological sensor 28 or medical information uploaded via the first port 25 or a second port 29.

As used herein, “computer readable medium” referred to as the storage medium 24 excludes any transitory signals, but includes any non-transitory data storage circuitry, e.g., buffers, cache, and queues, within transceivers of transitory signals.

A logic is at least partially stored in the non-transitory computer readable medium and that, when executed at least in part by a processor, causes the medical information bracelet 11 to transmit the physiological data detected from the physiological sensor 28 to the visual display screen 26, such that the visual display screen 26 displays blood pressure, heart rate, and other types of physiological data and receive medical information by the first port 25 or the second port 29. The processor stores the instructions of the medical information bracelet 11, such as sending information to the visual display screen 26 for display thereon.

The first port 25 is disposed on the middle section 15 of the elongated strap 12. In the illustrated embodiment, the first port 25 is disposed in the first surface 16 and is retractable for protection against liquids, dust, etc. and for convenience. The first port 25 is not centered in the middle section 15 in order to allow the visual display screen 26 to have the majority of the space on the middle section 15. In the illustrated embodiment, the first port 25 is a Universal Serial Bus (USB) port. However, in alternate embodiments, the first port 25 is a charging port. The first port 25 is configured to be in electronic communication with the storage medium 23 to allow users and medical professionals convenient access to the medical history of the user when retrieving the information while also providing convenient storing of information. This is extremely useful in an emergency or when the user is incapacitated and cannot convey the information themselves.

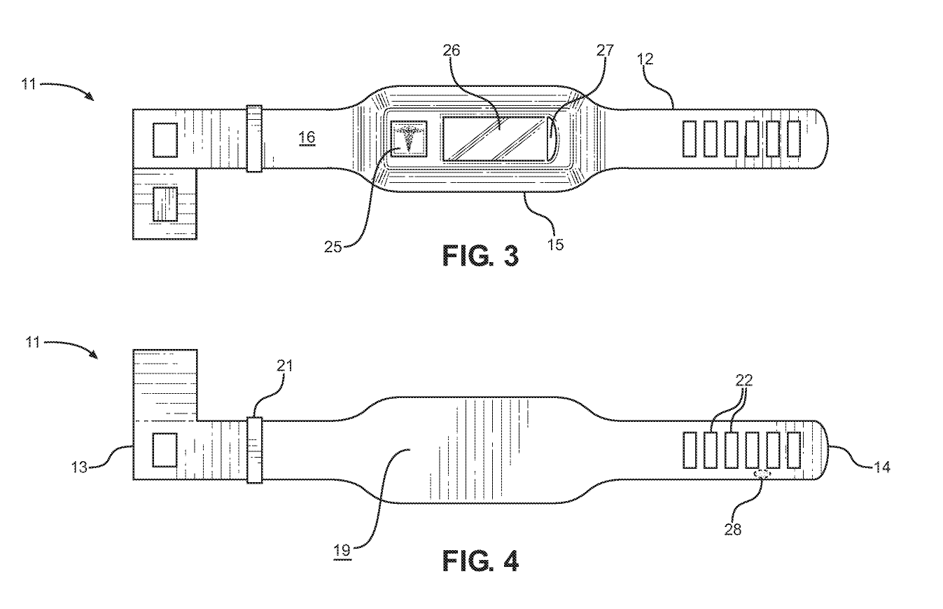

Referring now to FIG. 3, there is shown a top view of the medical information bracelet. The first surface 16 comprises the visual display screen 26. In the illustrated embodiment, the visual display screen 26 is slightly off centered on the middle section 15 on the first surface 16 to allow for optimal viewing of information displayed on the visual display screen 26 and to allow the first port 25 to be disposed in the middle section 15 as well. However, in alternate embodiments, the visual display screen 26 is centered directly in the middle section 15. The visual display screen 26 displays current physiological data such as blood pressure, heart rate, and other types of data. Further, the visual display screen 26 utilizes a bar code that is scannable to provide access to the individual medical information. The scanner is a configured to provide certain individuals, such as those with National Provider Identifier (NPI) numbers, to upload and download medical information to and from the medical information bracelet.

In the illustrated embodiment, the first port 25 is marked with an embossed medical indicium to alert a medical professional or emergency responder that the medical information bracelet 11 contains retrievable medical information therein. However, in alternate embodiments, the embossed medical indicium is on a different section of the elongated strap 12, such as between the first port 25 and the strap keeper 21, or elsewhere. In the illustrated embodiment, the first port 25 is rectangular in shape. However, in alternate embodiments, the first port 25 is circular.

In the illustrated embodiment, the function button 27 is disposed adjacent the visual display screen 26 due to their compatibility with each other. The function button 27 allows users to view different displays, information, and change the date and time.

Referring now to FIG. 4, there is shown a bottom view of the medical information bracelet. The fourth surface 19 remains uniform against the user's wrist and comprises the physiological sensor 28. In the illustrated embodiment, the physiological sensor 28 is disposed on the distal end of the second end 14 adjacent to the plurality of apertures 22 to allow for the physiological sensor 28 to be close to the radial pulse in the user's wrist. However, in alternate embodiments, the physiological sensor 28 is disposed on the distal end of the first end 13 adjacent to the strap keeper 21 in order for the physiological sensor 28 to be close to the radial pulse of the user. The physiological sensor 28 is in electronic communication with the storage medium which is in electronic communication with the visual display screen. In this way, the visual display screen transmits the physiological data of the user.

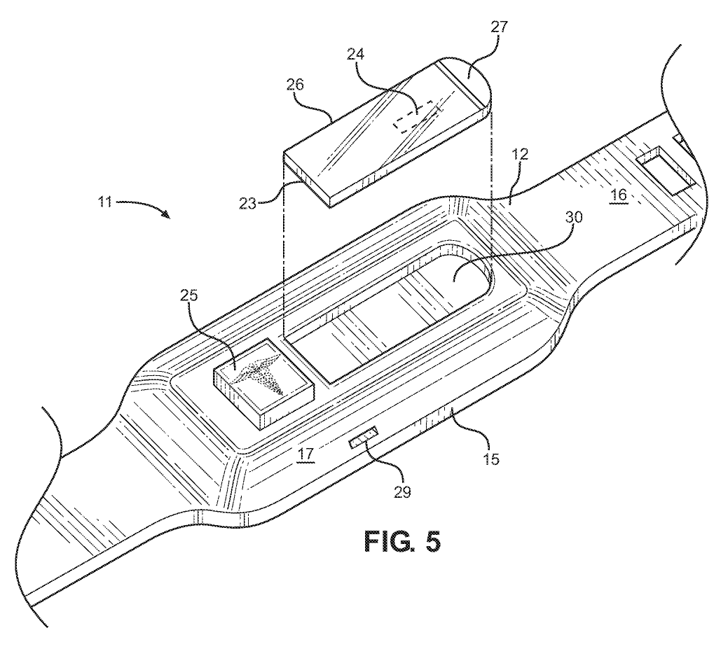

Referring now to FIG. 5, there is shown a perspective view of an alternate embodiment of the medical information bracelet. The function button 27 and the housing 23 are one piece in order to be removed together. In the illustrated embodiment, the housing 23 is removably connected to a recess 30 which is disposed within the middle section 15 on the first surface 16. The recess 30 is sized to receive the housing 23 flush within and the visual display screen 26 in the housing 23 is aligned flush with the first surface 16 when the housing 23 is disposed within the recess 30.

In this way, the elongated strap 12 can be interchangeable while avoiding having to replace the electrical components and the user's medical information stored thereon. An example of this would be if the first port 25 no longer functioned correctly, the user would remove the housing 23 which contains the storage medium 24, the visual display screen 26, and the function button 27 and place the housing 23 into a new elongated strap.

The second port 29 is disposed along the second surface 17. In the illustrated embodiment, the second port 29 is aligned with the visual display screen 26. However, in alternate embodiments, the second port 29 is disposed along any section of the second surface 17. In alternate embodiments, the second port 29 is disposed on any of the four surfaces. In the illustrated embodiment, the second port 29 is a micro Universal Serial Bus (USB) port. However, in alternate embodiments, the second port 29 is a charging port. Also in alternate embodiments, the first port 25 is a charging port, and the second port 29 is a USB port or vice versa. The second port 29 has a releasable cover to protect the second port 29 from liquids, dust, etc. The second port 29 is configured to be in electronic communication with the storage medium 24.

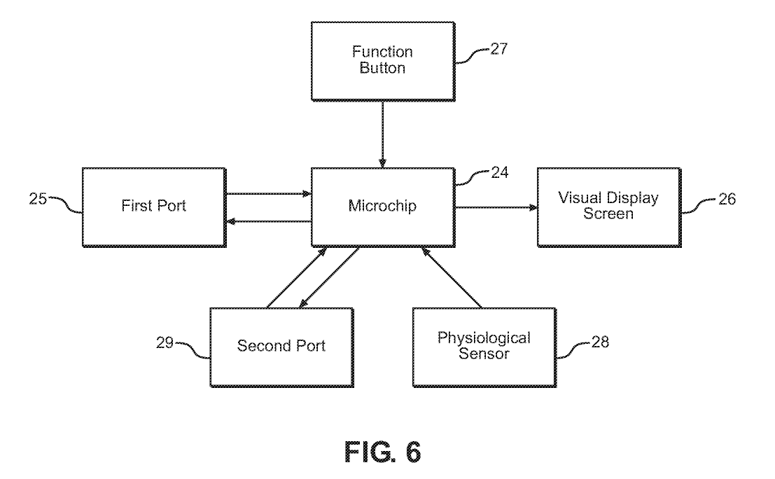

Referring now to FIG. 6, there is shown a block diagram of the electronic components of the medical information bracelet. A software application is stored on the microchip 24 that controls the functions of the device. The first port 25 sends and receives information onto the microchip 24. The second port 29 sends and receives information onto the microchip 24. A medical professional, preferably a person with a National Provider Identification, downloads the user's medical information to a USB thumb drive, or similar device, and uploads the data to a tablet or computer.

In the illustrated embodiment, the software is configured to be compatible and integrated with any existing software program on the tablet or computer, such that the medical information can be translated into any language. The physiological sensor 28 detects physiological data and inputs the data onto the microchip 24 which then displays the data on the visual display screen 26. The microchip 24 receives input from the function button 27 which then displays on the visual display screen 26.

In operation, the medical information bracelet and integrated software are configured to provide surveys, communicate with servers, and upload/download data thereto and therefrom. The system can automatically create a survey based on diagnosis or prescribed medications, and can send a message to the medical information band. For example, the user receives a new diagnosis of type 2 diabetes, the physician who prescribes the medication will require a two week follow up survey that will automatically be sent out to the user's medical information bracelet. This in turn will tell the user to log in to his or her account. There will be a five to ten question survey that identifies if the medication is working using ‘yes/no’ answers. If any question is answered with a ‘yes’ the survey will prompt the user to notify the MD. Thus, the MD will automatically receive information that will be generated by the user's access. It allows users and medical professionals easy access to the medical history of the user. This is extremely useful in an emergency or when the user is incapacitated and cannot convey the information themselves.

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A medical information bracelet, comprising:
an elongated strap;
a housing disposed on the elongated strap, wherein the housing comprises a visual display screen and a non-transitory computer readable medium;
a sensor configured to detect physiological data, wherein the sensor is in electronic communication with the visual display screen;
a port disposed on the elongated strap and operably connected to the non-transitory computer readable medium, wherein the port is configured to receive medical information;
a logic that is at least partially stored in the non-transitory computer readable medium and that, when executed at least in part by a processor, causes the medical information bracelet to perform a method, the method comprising:
transmitting the physiological data detected from the sensor to the visual display screen, such that the visual display screen displays blood pressure, heart rate, and other types of physiological data;
receiving medical information received by the port.

2) The medical information bracelet of claim 1, wherein a first end comprises a fastener which is a locking mechanism and a second end comprises a plurality of apertures to receive the fastener.

3) The medical information bracelet of claim 1, further comprising a middle section which is thicker and wider than the first end and the second end.

4) The medical information bracelet of claim 3, wherein the middle section comprises the port which is retractable within the middle section.

5) The medical information bracelet of claim 1, further comprising a medical indicium located on the port configured to notify an emergency responder that the medical information bracelet stores medical information.

6) The medical information bracelet of claim 1, wherein the elongated strap comprises a recess in the middle section that removably receives the housing and a function button.

7) The medical information bracelet of claim 6, wherein the function button is attached to the housing for removably inserting into the recess.

8) The medical information bracelet of claim 6, wherein the recess is sized to receive the housing and function button flush within and the visual display screen is aligned flush with a first surface when the housing is disposed within the recess.

9) The medical information bracelet of claim 6, wherein the function button allows users to view different displays, information, and change the date and time.
